(19) United States
(12) Patent Application Publication
(10) Pub. No.: US 2011/0003984 A1
(43) Pub. Date: Jan. 6, 2011

(54) BISPIDON LIGANDS AND THE METAL COMPLEXES THEREOF

(76) Inventors: Peter Comba, Wiesenbach (DE); Christina Haaf, Schriescheim (DE); Marion Kerscher, Edingen-Neckarhausen (DE); Achim Lienke, Schiedam (NL)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/668,784

(22) PCT Filed: Jun. 5, 2008
(86) PCT No.: PCT/EP2008/004506

§ 371 Date: Sep. 21, 2010

(30) Foreign Application Priority Data
Jul. 16, 2007 (DE) 10 2007 033 020.2

(51) Int. Cl. C07F 15/06; C07D 243/08; C07F 3/06; C07F 15/02; C07F 1/08

(52) U.S. Cl. 540/541; 540/575

(57) ABSTRACT

The present invention relates to novel bispidon ligands, a method for the production thereof, and the use thereof as a ligand in metal complexes and the selective separation of metals, metal complexes comprising said ligands, method for the production thereof, and the use of such metal complexes in organic synthesis, in bleaching, and in the radiopharmaceutical field.

The present invention relates to innovative bispidone ligands, to processes for preparing them, and to their use as ligands in metal complexes and in the selective separation of metals, to metal complexes comprising these ligands, to processes for preparing them, and to the use of such metal complexes in organic synthesis, in bleaching technology, and in the radiopharmaceutical sector.

Multidentate ligands form a group of compounds which is of great interest, since the possibilities for their use in areas both of industry and of medicine are diverse. By way of example, such ligands are employed for the selective separation of metal ions or in metal complexes for the catalytic oxidation of unsaturated compounds or for bleaching. Stable and biocompatible metal complexes also find application as contrast media or in the diagnosis and therapy of cancer diseases.

Many of these multidentate ligands and their metal complexes, however, have disadvantages in respect in particular of stability, reactivity, selectivity, and complexity of synthesis. Furthermore, the chemical possibilities for forming derivatives are inadequate for many of these ligands, meaning that the field of application, as a result of the consequent low flexibility, is relatively small.

It is an object of the present invention, therefore, to provide new bispidone ligands which can be used advantageously as ligands in metal complexes, and processes for preparing such ligands, and also metal complexes comprising these ligands.

This object is achieved by the embodiments of the present invention that are defined in the claims.

More particularly, a bispidone ligand of formula (1) is provided:

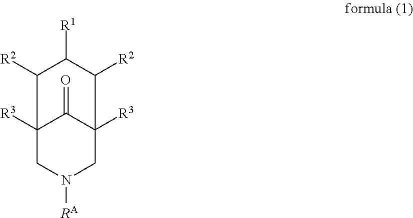

in which the radical R^{A} is selected from a group of one of the formulae (2a) to (2d):

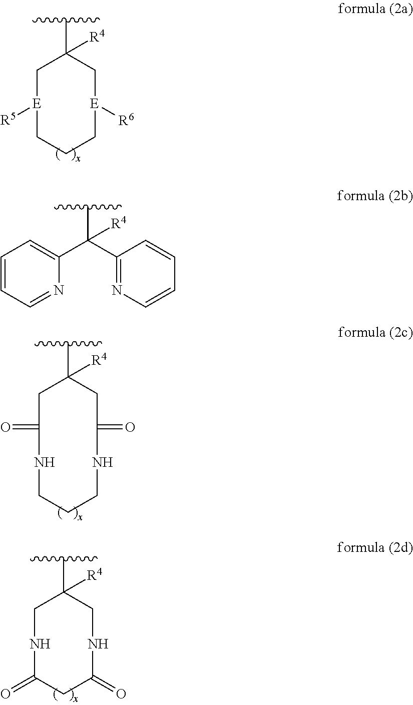

in which
E is selected from N or P,
x is an integer from 0 to 5,
the radical R¹ is selected from hydrogen, straight-chain or branched-chain (C₁₋₁₂) alkyl radicals, (C₃₋₈) cycloalkyl radicals, (C₅₋₁₂) aryl or heteroaryl radicals, (C₆₋₁₂) alkaryl or alkheteroaryl radicals, or a group of the formula (2a) to (2d) defined as above, with E and x being defined as above,
both radicals R² are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals,
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

The term “aryl radical” as used herein is not subject to any particular restriction and includes all chemical radicals which have an aromatic parent structure, such as a phenyl group, for example. In accordance with the present invention, the term “aryl radical” includes not only unsubstituted but also substituted aromatic groups.

The term “heteroaryl radical” as used herein is not subject to any particular restriction and includes all aromatic groups whose parent structure comprises one or more heteroatoms, such as a pyridyl radical, for example. Such groups may be, among others, derivatives of 5-membered rings, such as pyrroles, furans, thiophenes or imidazoles, or derivatives of 6-membered rings, such as pyrazines, pyridines or pyrimidines.

The term “alkaryl radical” as used in the present invention includes all those compounds which are substituted by at least one alkyl group, such as benzyl groups or ethylphenyl groups, for example. The “alkaryl radical” may be either unsubstituted or substituted on one or more alkyl groups and/or on the aromatic parent structure.

The term “alkheteroaryl radical” as used herein is not subject to any particular restriction and includes all compounds which comprise an aromatic parent structure having at least one heteroatom and at least one alkyl group. Alkheteroaryl radicals are exemplified, for example, by picolinyl radicals.

The term “carboxylic acid group or derivatives derived therefrom, selected from esters, amides, and peptides” as used herein stands for a corresponding —CO₂H, —CO₂⁻, —CONH₂, —CONHRₓ group, in which, in turn, Rₓ then stands for a corresponding amide or peptide radical.

In the formulae 2a, 2c, and 2d, as in the formulae 4a, 4c, and 4d, x is an integral value of 0 to 5. If x, for example, in the formula (2a)

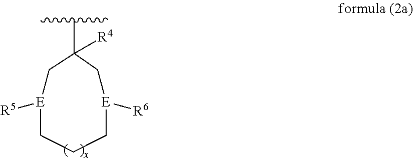

adopts the value 0, this means that there may be either a σ bond, and hence a cyclic compound of the formula (2a′)

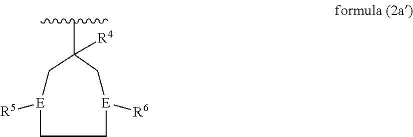

or the open-chain analog, i.e., an open-chain compound of the formula (2a″)

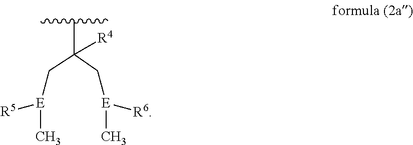

The same applies to the formula (2c). In the case of x=0 in formula (2c), the corresponding heptacycle compound or the corresponding open-chain analog may be present. In the case of formula (2d), for x=0 the compound present is the compound below of formula (2d″)

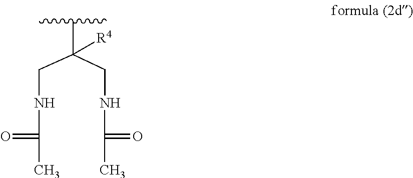

whereas if x=1 the compound present is the cyclic compound below:

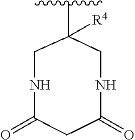

One preferred embodiment of the present invention relates to a bispidone ligand of formula (1), defined as above, in which the radical R^{A} is a group of the formula (2a):

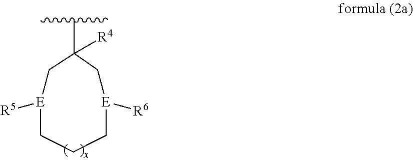

the radical R¹ is a straight-chain or branched-chain (C₁₋₆) alkyl radical, a (C₆₋₁₂) alkheteroaryl radical or a group of the above-defined formula (2a), both radicals R² are selected each independently of one another from hydrogen or a (C₆₋₁₂) aryl or (C₅₋₁₂) heteroaryl radical, both radicals R³ are selected each independently of one another from (C₁₋₆) aryl or heteroaryl groups or carboxylic acid groups or derivatives derived therefrom, selected from esters, amides, and peptides, the radical R⁴ is selected from hydrogen, straight-chain or branched-chain (C₁₋₁₂) alkyl radicals or (C₃₋₈) cycloalkyl radicals, and both radicals R⁵ and R⁶ are selected each independently of one another from hydrogen, straight-chain or branched-chain (C₁₋₁₂) alkyl radicals or (C₃₋₈) cycloalkyl radicals, and where E is selected from N or P, preferably from N, and x is an integer from 0 to 5.

In accordance with one specific embodiment of the present invention, a bispidone ligand defined as above is provided, in which the radical R^{A} is a group of the formula (2a):

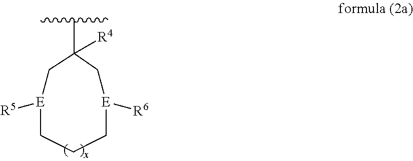

the radical R¹ is methyl, picolinyl or a group of the formula (3):

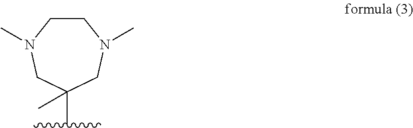

both radicals R² are hydrogen or pyridinyl groups, both radicals R³ are phenyl or methanoic acid methyl ester groups, and the radicals R⁴ to R⁶ are methyl, and where E is N and x=0, and the cyclic form of the compound is present.

In accordance with a further aspect of the present invention, a process for preparing one of the bispidone ligands of formula (1) defined as above is provided, where the radical R² represents hydrogen, comprising the steps of:

(a) the reacting of a compound of any of the formulae (4a) to (4d):

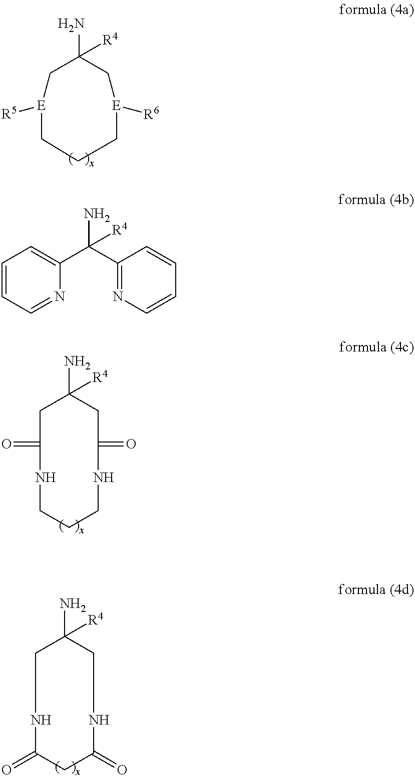

in which E is selected from N or P and x is an integer from 0 to 5, the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals,
with formaldehyde and with an acetone derivative of the formula (5):

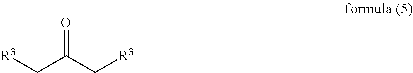

in which both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals and carboxylic acid groups or derivatives derived therefrom, selected from esters, amides, and peptides,
to form a piperidone intermediate of the formula (6):

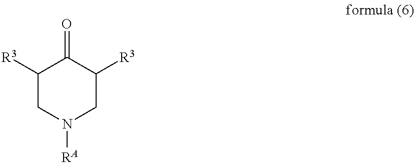

in which the radicals R^{A} and R³ are defined as above, and
(b) the reacting of the piperidone intermediate of the formula (6) with formaldehyde and with an amine of the general formula H₂N-R¹, in which the radical R¹ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, (C₆₋₁₂) alkyl-aryl or alkheteroaryl groups, or a group of the formula (2a) to (2d) defined as above, where E and x are defined as above, and where the radicals R³ to R⁶ are defined as above.

Another embodiment relates to a process for preparing one of the above-defined bispidone ligands of formula (1), in which the radical R¹ is a group of the formula (2a):

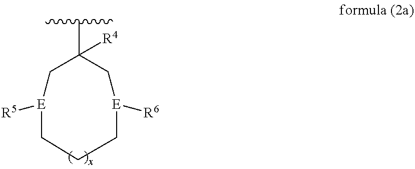

and both radicals R² are hydrogen, comprising reacting a compound of the formula (4a):

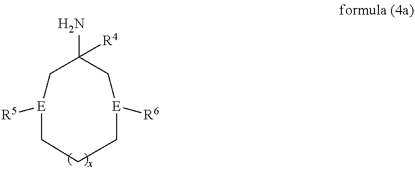

with formaldehyde and an acetone derivative of the formula (5):

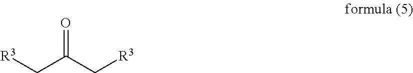

in which both radicals R³ each independently of one another are selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and carboxylic acid groups, or derivatives derived from them, selected from esters, amides, and peptides, the radicals R⁴ to R⁶ are selected in each case independently of one another from hydrogen or straight-chain or branched-chain (C₁₋₈) alkyl radicals, and where E is selected from N or P, and x=0 to 5.

In accordance with a further embodiment, a process for preparing one of the above-defined bispidone ligands of the formula (1) is provided, comprising the reacting of a compound of one of the above-defined formulae (4a) to (4d), in which E is selected from N or P, and x is an integer from 0 to 5, the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals and optionally both radicals R⁵ and R⁶ each independently of one another are selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, with formaldehyde and with a compound of the formula (7):

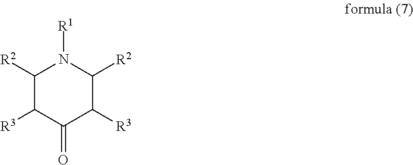

in which the radical R¹ is selected from hydrogen, straight-chain or branched-chain (C₁₋₁₂) alkyl radicals, (C₃₋₈) cycloalkyl radicals, (C₅₋₁₂) aryl or heteroaryl radicals, (C₆₋₁₂) alkaryl or alkheteroaryl groups, or a group of the formula (2a) to (2d) as defined above, where E and x are defined as above, both radicals R² are selected each independently of one another from hydrogen, straight-chain or branched-chain (C₁₋₁₂) alkyl radicals, (C₃₋₈) cycloalkyl radicals, and (C₆₋₁₂) aryl or (C₅₋₁₂) heteroaryl radicals, both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides, the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

The present invention further relates to the use of the above bispidone ligands for the selective separation of metal ions, for the preparation of metal complexes for the catalytic oxidation of unsaturated compounds, for catalytic bleaching or for the diagnosis and/or therapy of tumor diseases.

The term “selective separation of metals” as used herein is not subject to any particular restriction and relates to all applications in which there are at least two metals, of which one or more are to be accumulated and/or removed.

The term “catalytic oxidation” includes all those reactions which introduce an oxygen atom or oxygen molecule into a target compound and/or by means of which the oxidation numbers of the participating reactants increase or decrease.

Furthermore, the term “tumor diseases” includes all those diseases of a mammal which relate to a direct cancer disease or are in any way associated with a cancer disease.

A further aspect of the present invention relates to a metal complex comprising one of the above-defined bispidone ligands, in which the metal is selected from Mn, Cu, Fe, Co, Ti, V, Mo, W, Tc, In, Ga, Y, Re or the rare earth metals.

The term “metal” as used herein is not subject to any particular restriction, and includes the metal as such and also its ions in all known oxidation states.

According to one specific embodiment of the present invention, the metal in a metal complex of the invention of this kind is a radioactive nuclide.

The term “nuclide” as used herein encompasses all isotopes of the aforementioned metals that can be used in accordance with the invention. The term “nuclide” encompasses more particularly those metal isotopes which are used preferentially in radiopharmaceutical compounds, such as ^{99m}Tc, ⁶⁴Cu (for example, for positron emission tomography), ⁶⁷Cu (for example, for use in therapy), ⁸⁶Y, ⁹⁰Y, and ¹⁸⁸Re, for example.

A further aspect of the present invention relates to a process for preparing one of the above metal complexes, in which the bispidone ligand defined as above is reacted with a metal salt solution of the corresponding metal at a temperature in the range from 20 to 100° C.

In accordance with a further aspect, the present invention relates to the use of the metal complex as defined above in the catalytic oxidation of unsaturated compounds, in catalytic bleaching, and in the diagnosis and/or therapy of tumor diseases.

The present invention further provides a compound having the formula (6):

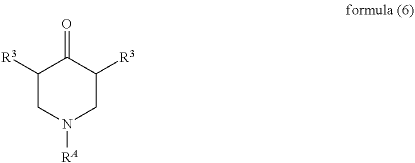

in which the radical R^{A} is selected from a group of one of the formulae (2a) to (2d):

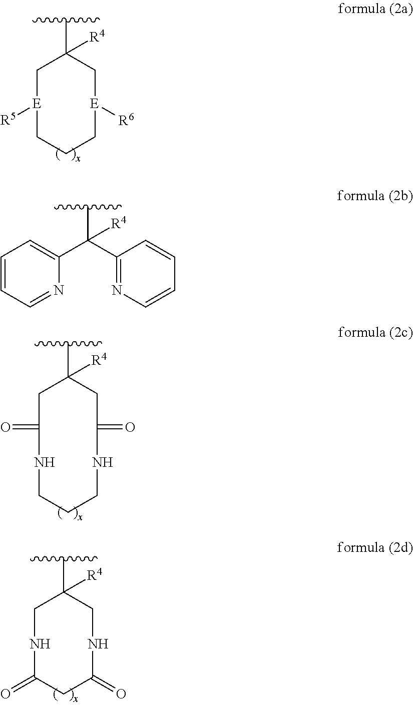

in which E is selected from N or P, and x is an integer from 0 to 5,
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

Advantageous features of the bispidone ligands of the present invention are that, on the basis of their variable structure, it is possible to encompass a multiplicity of very highly efficient metal complexes, for the purpose, for example, of olefin oxidation, in applications as bleaching agents, or in the diagnosis and/or therapy of tumor diseases. The property of the metal complex in question may already be influenced in a targeted way through the synthesis of the bispidone ligands of the invention. Furthermore, surprisingly, the present invention provides diverse synthesis pathways which advantageously allow the preparation of the bispidone ligands.

The purpose of the examples which follow is to illustrate the present invention further, without restricting it in any way whatsoever.

### EXAMPLES

### Example 1

### Preparation and Characterization of 1-(1,4,6-trimethyl-1,4-diazacycloheptan-6-yl)-3,5-diphenylpiperidin-4-one (P1)

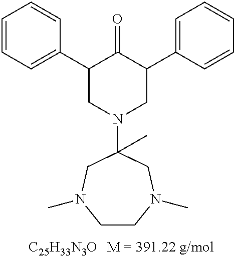

### Batch:

| 1,4,6-trimethyl-6-amino-1,4- | 25.50 | mmol (4.00 g) [~4 eq.] |
|---|---|---|
| diazacycloheptane: | | |
| 37% formalin: | 25.50 | mmol (2.0 ml) [~4 eq.] |
| 1,3-diphenylpropan-2-one: | 12.25 | mmol (2.68 g) [~2 eq.] |
| HOAc: | 4.4 | ml eq. [~3 eq.] |
| DME: | 20 | ml |
| KOH solution: | 25 | ml |
| diethyl ether: | 500 | ml |

### Procedure

In a 250 ml three-neck flask, the 1,4,6-trimethyl-6-amino-1,4-diazacycloheptane in solution in 10 ml of DME is admixed at 0° C. with glacial acetic acid and formalin. Subsequently, 1,3-diphenylpropan-3-one in solution in 10 ml of DME is admixed dropwise. The mixture is heated at 90° C. with stirring for 6 hours. After the mixture has cooled to room temperature, the solvent and water formed are removed under reduced pressure. The orange oil is then taken up in diethyl ether and admixed with concentrated perchloric acid. White crystals precipitate, which are isolated by filtration and purified with a hot 1:1 water/ethanol mixture. Drying of the crystals under a high vacuum results in the simple perchlorate salt of piperidone. The free piperidone is obtained by adding 20% KOH solution and carrying out extraction with diethyl ether. Subsequent removal of the solvent under reduced pressure gives the desired product.

Yield: 2.45 g (4.99 mmol, 40.73%) as perchlorate salt; 1.31 g (3.35 mol, 67.31%) as free piperidone; total yield 27.35%.

Appearance: white crystals.

¹H-NMR: (CD₃CN, 200.13 MHz, as perchlorate salt)

δ=0.98 ppm (s, 3H, —CH₃); δ=2.60 ppm (s, 6H, —CH₃); δ=2.76-3.40 ppm (m, 12H, —CH₂); δ=4.19 ppm (dd, ³J₁=12 Hz, ³J₂=6 Hz, 2H, —CH); δ=7.22-7.40 ppm (m, 10H, —CHₐᵣₒₘₐₜ).

¹³C-NMR: (CD₃CN, 50.33 MHz, as perchlorate salt)

δ=15.06 ppm (1C, —CH₃); δ=45.91 (2C, —CH₃); δ=52.89 ppm (2C, C_{aromatic}—C—CH₂); δ=54.67 ppm (2C, CH₂—CH₂), δ=56.01 ppm (2C, —CH); δ=58.05 ppm (1C, —C); δ=62.80 ppm (2C, C—CH₂—N); δ=126.91 (2C, —CH_{aromatic/para}); δ=128.00 ppm (4C, —CH_{aromatic/ortho}); δ=129.10 ppm (4C, —CH_{aromatic/meta}); δ=136.50 ppm (2C, —C_{aromatic}); δ=205.76 ppm (1C, —CO).

IR spectrum: (KBr disk):

\tilde{v}=3419 cm⁻¹ (b) (OH in H bonds); \tilde{v}=3087 cm⁻¹, 3059 cm⁻¹, 3027 cm⁻¹, (m, aryl-CH stretching); \tilde{v}=2967 cm⁻¹, 2963 cm⁻¹, 2843 cm⁻¹ (s)(CH₂ and CH₃ stretching); \tilde{v}=2801 cm⁻¹ (s) (N—CH₃ and N—CH₂ (Bohlmann band) stretching); \tilde{v}=1717 cm⁻¹ (s) (CO vibration); \tilde{v}=1599 cm⁻¹, 1496 cm⁻¹ (m) (aryl-C═C stretching); \tilde{v}=1452 cm⁻¹ (s) (CH₂ and CH₃ bending); \tilde{v}=1376 cm⁻¹ (m) (CH₃ bending, symmetric), \tilde{v}=760 cm⁻¹, 748 cm⁻¹ (w) (CH bending, mono-substitution).

Mass spectrum (EI⁺):

m/z: 391.3 [P1]⁺ (9%); 321.3 [N(C(CH₃)₂CH₂CH₃)—(CH₂CHPh)₂CO)]⁺ (41%); 99.1 [N(CH₂)(CH₂C(CH₃)—(CH₂NCH₃)+H⁺]⁺ (100%); 57.1 [N(CH₃)(CH₂)(CH₂)]⁺ (40%); 42.1 [N(CH₂)₂]⁺, [CH₃CHCH₂]⁺ (22%).

Elemental Analysis:

calculated: C, (61.03%); H, (6.97%); N, (8.54%); Cl, (7.21%).

found: C, (60.95%); H, (6.94%); N, (8.56%); Cl, (6.99%).

### Example 2

### Preparation and Characterization of 1,5-diphenyl-3-methyl-7-(1,4,6-trimethyl-1,4-diazacycloheptan-6-yl)diazabicyclo[3.3.1]nonan-9-one (B1)

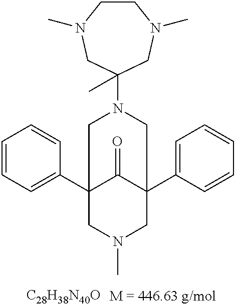

### Batch:

| 1-(1,4,6-trimethyl-1,4- | 3.02 | mmol (1.18 g) [~1 eq.] |
|---|---|---|
| diazacycloheptan-6-yl)-3,5- | | |
| diphenylpiperidin-4-one (P1): | | |
| 37% formalin: | 6.64 | mmol (0.50 ml) [~2.2 eq.] |
| 41% methylamine: | 3.02 | mmol (0.25 ml) [~1 eq.] |
| HOAc: | 0.7 | ml [~4 eq.] |
| Methanol: | 12 | ml |
| KOH solution (conc.): | 100 | ml |
| Diethyl ether: | 1000 | ml |

### Procedure:

In a 250 ml three-neck flask, at 0° C., methylamine, formalin, and acetic acid are introduced in 6 ml of methanol. Subsequently, at room temperature, the piperidone, in solution in 6 ml of methanol, is added. The mixture is stirred at 65° C. for 8 hours. It is subsequently evaporated to dryness and the residue is taken up with water. Potassium carbonate is added slowly to a pH of approximately 9. The aqueous solution is extracted with diethyl ether. The solvent is removed under reduced pressure. Purification is carried out by recrystallization from methanol.

Yield: 1.18 g (2.64 mmol, 87.4%).

Appearance: white crystals.

¹H-NMR: (CDCl₃, 200.13 MHz)

δ=1.14 ppm (s, 3H, —CH₃); δ=2.21 ppm (m, 6H, —N—CH₃); δ=2.22 ppm (d, ²J=13.4 Hz, 2H, —C—N—CH×₂ₐₓ—C); δ=2.41 ppm (m, 3H, —N—CH₃); δ=2.45 ppm (m, 4H, CH₂—CH₂); δ=2.78 ppm (d, ²J=13.4 Hz, 2H, —C—N—CH₂ₐₓ—C); δ=2.99 ppm (d, ²J=10.6 Hz, 2H, —CH₂ₐₓ—N—CH₃/—CH₂ₐₓ—C); δ=3.25 ppm (d, ²J=11.0 Hz, 2H, —N—CH₂ₐₓ—C/—CH₂ₐₓ—C—CH₃); δ=3.41 ppm (d, ²J=10.6 Hz, 2H, —CH_{2eq}—N—CH₃/—N—CH_{2eq}—C); δ=3.79 ppm (d, ²J=11.0 Hz, 2H, —N—CH_{2eq}—C/—CH_{2eq}—N—CH₃); δ=7.24 ppm (m, 10H, —CH_{aromatic}).

¹³C-NMR: (CDCl₃, 50.33 MHz)

δ=18.85 ppm (1C, —C—CH₃); δ=45.97 (1C, —N—CH₃); δ=49.27 ppm (2C, —CH₃); δ=55.80 ppm (1C, —C—CH₃), δ=59.20 ppm (2C, —C—C_{aromatic}); δ=59.99 ppm (2C, —N—CH₂—C); δ=62.56 ppm (2C, N—CH₂); δ=66.67 ppm (2C, —N—CH₂—C/—CH₂—N—CH₃); δ=67.36 ppm (2C, —N—CH₂—C/—CH₂—N—CH₃); δ=126.87 (2C, —CH_{aromatic/para}); δ=127.32 ppm (4C, —CH_{aromatic/ortho}); δ=128.27 ppm (4C, —CH_{aromatic/para}); δ=143.51 ppm (2C, —C_{aromatic}); δ=212.15 (1C, CO).

IR spectrum: (KBr disk):

\tilde{v}=3022 cm⁻¹ (m) (aryl-CH stretching); \tilde{v}=2937 cm⁻¹, (s) (CH₂ and CH₃ stretching); \tilde{v}=2802 cm⁻¹ (s) (N—CH₃ and N—CH₂ (Bohlmann band) stretching); \tilde{v}=1733 cm⁻¹ (s) (CO vibration), \tilde{v}=1599 cm⁻¹, 1575 cm⁻¹, 1495 cm⁻¹ (m) (aryl-C═C stretching); \tilde{v}=1456 cm⁻¹ (s) (CH₂ and CH₃ bending); \tilde{v}=1320 cm⁻¹ (m) (CH₃ bending, symmetric), \tilde{v}=761 cm⁻¹, 693 cm⁻¹ (w)(CH bending, mono-substitution).

Mass spectrum (FAB⁺):

m/z: 447.4 [B1H⁺]⁺ (100%), 141.1 [N(CH₃)(CH₂C(CH₃)(CH₂NH—(CH₃)CH₂CH₂))]+ (62%).

Elemental Analysis:

calculated: C, (75.30%); H, (8.58%); N, (12.54%).

found: C, (75.00%); H, (8.53%); N, (12.52%).

### Example 3

### Preparation and Characterization of [Cu^{II}(B1)(NCCH₃)](BF₄)₂(CuB1(NCCH₃)

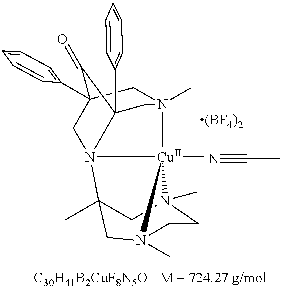

### Batch:

| 1,5-Diphenyl-3-methyl-7-(1,4,6- | 0.67 | mmol (299 mg) [1 eq.] |
|---|---|---|
| trimethyl-1,4-diazacycloheptan-6- | | |
| yl)diazabicyclo[3.3.1]nonan-9-one | | |
| (B1): | | |
| Cu^{II}(BF₄)₂(H₂O)₆ | 0.67 | mmol (232 mg) [1 eq.] |
| Acetonitrile: | 22 | ml |
| Diethyl ether: | 50 | ml |

### Procedure:

In a 250 ml three-neck flask, copper^{II} tetrafluoroborate is introduced in 5 ml of acetonitrile. The bispidone is also dissolved in 17 ml of acetonitrile and added to the dissolved copper tetrafluoroborate. The solution, which is now dark blue, is heated at 82° C. for 5 minutes. After it has cooled to room temperature, crystallization is initiated by ether diffusion. Purification was carried out by further recrystallization from acetonitrile.

Yield: 271 mg (0.37 mmol, 55.2%).

Appearance: blue crystals.

IR spectrum: (KBr disk):

\tilde{v}=3450 cm⁻¹ (b) (H₂O in disk); \tilde{v}=3064 cm⁻¹ (w), 3029 cm⁻¹ (w)(aryl-CH stretching); \tilde{v}=2977 cm⁻¹ (m) D 2935 cm⁻¹ (m) (CH₂ and CH₃ stretching); \tilde{v}=2248 cm⁻¹ (s)(C≡N stretching); \tilde{v}=1742 cm⁻¹ (s) (CO vibration); v=1635 cm⁻¹ (m), 1505 cm⁻¹ (m)(aryl-C═C stretching); \tilde{v}=1479 cm⁻¹ (m), 1450 cm⁻¹ (m) (CH₂ and CH₃ bending); \tilde{v}=1048 cm⁻¹ (s) (BF vibration); \tilde{v}=766 cm⁻¹ (m), 699 cm⁻¹ (s)(CH bending, mono-substitution).

UV-Vis spectroscopy: (CH₃CN)

λ₁=903 nm (ε=341 l/(mol·cm)), λ₂=627 nm (ε=684 l/(mol·cm)).

ESR spectroscopy: (Acetonitrile:toluene 1:1)

g_{¦}=2.21; g₋=2.08; A_{¦}=127 G; A₋=2 G.

Cyclic voltammogram: (vs. Ag/AgNO₃, T=25° C. in CH₃CN)

E_{1/2}=−377 mV.

Mass spectrum: (ESI)

m/z: 568.4 [CU^{II}(B1)(NCCH₃)(H₃O)]⁺ (100%); 509.5 [Cu^{II}(B1)(H)]⁺ (6%); 275.4 [Cu^{II}(B1)(NCCH₃)]²⁺ (62%); 255.0 [Cu^{II}(B1)]²⁺ (14%).

Elemental Analysis: CuB1(NCCH₃)

calculated: C, (49.71%); H, (5.70%); N, (9.66%).

found: C, (49.72%); H, (5.75%); N, (10.75%).

### Example 4

### Preparation and Characterization of 1,5-diphenyl-3-(2-picolylamine)-7-(1,4,6-trimethyl-1,4-diazacycloheptan-6-yl)diazabicyclo[3.3.1]nonan-9-one (B2)

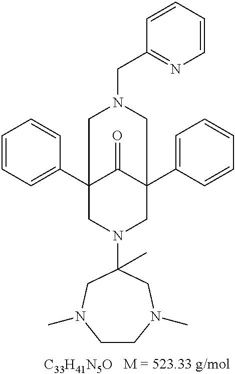

### Batch:

| 1-(1,4,6-Trimethyl-1,4- | 1.28 | mmol (500 mg) [~1 eq.] |
|---|---|---|
| diazacycloheptan-6-yl)-3,5- | | |
| diphenylpiperidin-4-one (P1): | | |
| 37% formalin: | 2.82 | mmol (0.22 ml) [~2.2 eq.] |
| 2-Picolylamine: | 1.28 | mmol (0.13 ml) [~1 eq.] |
| HOAc: | 0.29 | ml [~4 eq.] |
| Methanol: | 10 | ml |
| KOH solution (conc.): | 100 | ml |
| Diethyl ether: | 1000 | ml |

### Procedure:

In a 100 ml three-neck flask, at 0° C., picolylamine, formalin and acetic acid are introduced in 6 ml of methanol. Subsequently, at room temperature, the piperidone, in solution in 6 ml of methanol, is added. The mixture is stirred at 65° C. for 4 hours. It is then evaporated to dryness and the residue is taken up with water. Potassium carbonate is added slowly until the pH is approximately 9. The aqueous solution is extracted with diethyl ether. The solvent is removed under reduced pressure. Purification is carried out by recrystallization from methanol.

Yield: 455.3 mg (0.87 mmol, 68%).

Appearance: colorless crystals.

¹H-NMR: (CDCl₃, 200.13 MHz)

δ=1.19 ppm (s, 3H, —CH₃); δ=2.29 ppm (m, 6H, —N—CH₃); δ=2.31 ppm (d, ²J=13.4 Hz, 2H, —C—N—CH₂ₐₓ); δ=2.48 ppm (m, 4H, —CH₂—CH₂); δ=2.85 ppm (d, ²J=13.4 Hz, 2H, —C—N—CH₂ₐₓ); δ=3.20 ppm (d, ²J=10.8 Hz, 2H, —CH₂ₐₓ—N—CH₂—Py); δ=3.28 ppm (d, ²J=11.0 Hz, 2H, —N—CH₂ₐₓ—C); δ=3.61 ppm (d, ²J=10.8 Hz, 2H, —CH_{2eq}—N—CH₂—Py); δ=3.80 ppm (d, ²J=11.0 Hz, 2H, —N—CH_{2eq}—C); δ=3.89 ppm (1H, —CH₂—Py); δ=3.92 ppm (1H, —CH₂-Py); δ=7.26 ppm (m, 10H, —CH_{aromatic}); δ=7.67 ppm (m, 3H, CH_{3,4,5-pyridine}); δ=8.55 ppm (m, 1H, —CH_{6-pyridine})

¹³C-NMR: (CDCl₃, 50.33 MHz)

δ=25.90 ppm (1C, —CH₃); δ=48.54 ppm (2C, —CH₃); δ=54.39 ppm (1C, —C—CH₃); δ=58.87 ppm (2C, C—CH₂—N) δ=59.85 ppm (2C, —C—C_{aromatic}); δ=61.79 ppm (2C, —CH₂—CH₂); δ=63.36 ppm (1C, —CH₂—Py); δ=64.92 ppm (2C, —l N—CH₂—C); δ=65.83 ppm (2C, —CH₂—N—CH₂—Py); δ=122.15 ppm (1C, —C_{pyridine,ortho}) δ=123.01 ppm (1C, —C_{pyridine,ortho}); δ=126.47 ppm (2C, —CH_{aromatic/para}); δ=126.59 ppm (4C, —CH_{aromatic/ortho}) δ=127.51 ppm (4C, —CH_{aromatic/meta}); δ=136.30 ppm (1C, —CH—CH_{pyridine/meta}) δ=142.96 ppm (2C, —C_{aromatic}); δ=148.73 ppm (1C, —N—CH_{pyridine/ortho}); δ=167.77 ppm (1C, C_{pyridine}).

IR spectrum: (KBr disk):

\tilde{v}=3409 cm⁻¹ (b) (H₂O in disk); \tilde{v}=3058 cm⁻¹ (m) (aryl-CH stretching); \tilde{v}=2938 cm⁻¹ (s) (CH₂ and CH₃ stretching); \tilde{v}=2807 cm⁻¹ (s) (N—CH₃ and N—CH₂ (Bohlmann band) stretching); \tilde{v}=1720 cm⁻¹ (s) (CO vibration); \tilde{v}=1589 cm⁻¹ (s), 1570 cm⁻¹ (s), 1497 cm⁻¹ (m) (aryl-C═C stretching); \tilde{v}=1474 cm⁻¹ (s), 1446 cm⁻¹ (s), 1433 cm⁻¹ (s) (CH₂ and CH₃ bending); \tilde{v}=1360 cm⁻¹ (m) (CH₃ bending, symmetric) \tilde{v}=759 cm⁻¹ (s), 698 cm⁻¹ (s) (CH bending, mono-substitution).

Mass spectrum: (MALDI-TOF)

m/z: 573.8 (70%) [B2 (H₃O⁺)(CH₃OH)]⁺, 524.7 (100%) [B2 (H⁺)]⁺, 141.6 (57%) [A1-NH₂⁻)]⁺

### Example 5

### Preparation and Characterization of [Cu^{II}(B2)](BF₄)₂ (CuB2)

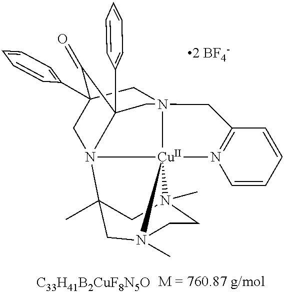

### Batch:

| 1,5-Diphenyl-3-(2-picolylamine)-7- | 0.12 | mmol (65 mg) [1 eq.] |
|---|---|---|
| (1,4,6-trimethyl-1,4- | | |
| diazacycloheptan-6- | | |
| yl)diazabicyclo[3.3.1]nonan-9-one | | |
| (B2): | | |
| Cu^{II}(BF₄)₂(H₂O)₆ | 0.12 | mmol (27 mg) [1 eq.] |
| Acetonitrile: | 10 | ml |
| Diethyl ether: | 50 | ml |

### Procedure:

In a 100 ml three-neck flask, copper tetrafluoroborate is introduced in 5 ml of acetonitrile. The bispidone is also dissolved in 5 ml of acetonitrile and added to the dissolved copper tetrafluoroborate. The solution, which is now dark blue, is heated at 82° C. for 5 minutes. After it has cooled to room temperature, crystallization is initiated by ether diffusion. Purification is carried out by recrystallization from acetonitrile.

Yield: 54.8 mg (0.07 mmol, 58.3%).

Appearance: blue crystals.

IR spectrum: (KBr disk):

\tilde{v}=3502 cm⁻¹ (b) (H₂O in disk); \tilde{v}=3060 cm⁻¹, (w) (aryl-CH stretching); \tilde{v}=2845 cm⁻¹ (w) (CH₂ and CH₃ stretching); \tilde{v}=1743 cm⁻¹ (m) (CO vibration); v=1616 cm⁻¹ (m), 1505 cm⁻¹ (w)(aryl-C═C stretching); v=1448 cm^{l} (m) (CH₂ and CH₃ bending); \tilde{v}=1060 cm⁻¹ (s) (BF vibration); \tilde{v}=765 cm⁻¹ (m), 701 cm⁻¹ (m)(CH bending, mono-substitution).

UV-Vis spectroscopy: (CH₂CN)

λ₁=913 nm (ε=198 l/(mol·cm)), λ₂=608 nm (ε=280 l/(mol·cm)).

ESR spectroscopy: (Acetonitrile:toluene 1:1)=

g_{¦}=2.43; gₓ=2.09; g_{y}=2.06; A_{¦}=120 G; Aₓ=14 G;

A_{y}=33 G.

Cyclic voltammogram: (vs. Ag/AgNO₃, T=25° C. in CH₃CN)

E_{1/2}=−536 mV.

Mass spectrum: (ESI)

m/z: 586.4 (4%) [Cu^{II}(B2)(H)]⁺; 293.4 (100%) [Cu^{II}(B2)]²⁺

Elemental Analysis: CuB2(H₂O)

calculated: C, (50.89%); H, (5.56%); N, (8.99%).

found: C, (50.62%); H,(5.53%); N,(9.09%).

### Example 6

### Preparation and Characterization of 1,5-diphenyl-3,7-(di[1,4,6-trimethyl-1,4-diazacycloheptan-6-yl)diazabicyclo[3.3.1]nonan-9-one (B3)

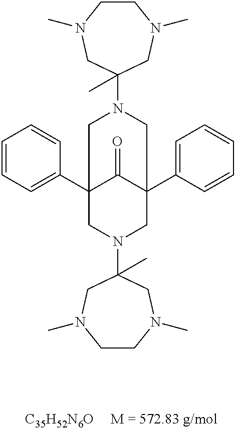

### Batch:

| 1,4,6-Trimethyl-6-amino-1,4- | 10.2 | mmol (1.60 g) [~2 eq.] |
|---|---|---|
| diazacycloheptan: | | |
| 37% formalin: | 20.5 | mmol (1.6 ml) [~4 eq.] |
| 1,3-Diphenylpropan-2-one: | 5.0 | mmol (1.07 g) [~1 eq.] |
| HOAc: | 1.6 | ml [~6 eq.] |
| THF: | 20 | ml |
| KOH solution (conc.): | 100 | ml |
| Diethyl ether: | 1000 | ml |
| Methanol: | 25 | ml |

### Procedure:

In a 100 ml flask, at 0° C., amine A1, formalin, and acetic acid are introduced in 15 ml of THF. Subsequently, at room temperature, the 1,3-diphenylpropan-2-one, in solution in 5 ml of THF, is added. The mixture is stirred at 65° C. for 29 hours. It is evaporated to dryness, then admixed with concentrated KOH solution and extracted with diethyl ether. The solvent is removed under reduced pressure. Subsequently the oily residue is taken up in a little methanol and admixed slowly and with stirring with water. A white solid is precipitated. This solid is isolated, dried, and recrystallized from methanol.

Yield: 1.78 g (3.10 mmol, 62.0%).

Appearance: colorless crystals.

¹H-NMR: (CDCl₃, 200.13 MHz)

δ=1.15 ppm (s, 6H, —CH₃); δ=2.20-2.25 ppm (m, 16H, —N—CH₃, —C—N—CH₂ₐₓ); δ=2.35-2.57 ppm (m, 8H, —CH₂—CH₂); δ=2.82 ppm (d, ²J=12.6 Hz, 4H, —C—N—CH_{2eq}); δ=3.24 ppm (d, ²J=11.0 Hz, 4H, —N—CH₂ₐₓ—C); δ=3.75 ppm (d, ²J=11.0 Hz, 4H, —N—CH_{2eq}—C) δ=7.26 ppm (m, 10H, —CH_{aromatic}).

¹³C-NMR: (CDCl₃, 50.33 MHz)

δ=25.61 ppm (2C, —CH₃); δ=48.84 (4C, —CH₃); δ=54.86 ppm (2C, —C—CH₃); δ=58.72 ppm (4C, C—CH₂—N), δ=60.15 ppm (4C, —C—C_{aromatic}); δ=62.11 ppm (4C, —CH₂—CH₂); δ=66.10 ppm (4C, —N—CH₂—C); δ=126.03 ppm (2C, —CH_{aromatic/para}); δ=126.91 ppm (4C, —CH_{aromatic/ortho}); δ=127.75 ppm (4G, —CH_{aromatic/meta}); δ=144.23 ppm (2C, —C_{aromatic}); δ=212.50 ppm (1C, —CO).

IR spectrum: (KBr disk):

\tilde{v}=3410 cm⁻¹ (b) (H₂O in disk); \tilde{v}=3048 cm⁻¹, \tilde{v}=3027 cm⁻¹ (m) (aryl-CH stretching); \tilde{v}=2936 cm⁻¹ (s) (CH₂ and CH₃ stretching); \tilde{v}=2799 cm⁻¹ (s) (N—CH₃ and N—CH₂ (Bohlmann band) stretching); \tilde{v}=1716 cm⁻¹ (s) (CO vibration); \tilde{v}=1601 cm⁻¹ (w), 1498 cm⁻¹ (m) (aryl-C═C stretching); \tilde{v}=1460 cm⁻¹ (s) (CH₂ and CH₃ bending); v=1373 cm⁻¹ (m) (CH₃ bending, symmetric) \tilde{v}=717 cm⁻¹ (s), 698 cm⁻¹ (s)(CH bending, mono-substitution).

Mass spectrum (ESI, MeOH)

m/z: 605.6 (19%) [B3(CH₃OH)(H)]⁺; 573.5 (100%) [B3(H)]⁺.

### Example 7

### Preparation and Characterization of 1,5-dimethylcarboxyl-6,8-dipyridyl-7-methyl-3-[1,4,6-trimethyl-1,4-diazacycloheptan-6-yl]diazabicyclo-[3.3.1]nonan-9-one (B4)

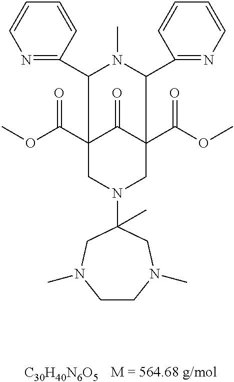

### Batch:

| 1,4,6-Trimethyl-6-amino-1,4- | 1.2 | mmol (188 mg) [~1.2 eq.] |
|---|---|---|
| diazacycloheptan: | | |
| 37% formalin: | 1.3 | mmol (0.1 ml) [~1.3 eq.] |
| 1,3-Dimethylcarboxyl-2,6- | 1.0 | mmol (387 mg) [~1 eq.] |
| dipyridyl-I-methylpiperidin-4-one | | |
| P2: | | |
| THF: | 10 | ml |
| Methanol: | 25 | ml |

### Procedure:

In a 100 ml three-neck flask, at 0° C., amine A1 and formalin are introduced in 5 ml of THF. Subsequently, at room temperature, P2 in 5 ml of THF is added. The mixture is stirred at room temperature for 30 hours. It is evaporated to dryness and the oily residue is recrystallized from methanol.

Appearance: colorless crystals.

¹H-NMR: (CDCl₃, 200.13 MHz)

δ=0.98 ppm (s, 3H, —CH₃); δ=2.00 ppm (s, 3H, —N—CH₃); δ=2.15 ppm (m, 8H, —NCH₃, —N—CH₂,x—C); δ=2.36 ppm (m, 4H, —CH₂—CH₂); δ=2.62 ppm (d, ²J=13.8 Hz, 2H, —N—CH_{2eq}—C); δ=3.06 ppm (d, ²J=12.2 Hz, 2H, —N—CH₂ₐₓ—C); δ=3.42 ppm (d, ²J=11.0 Hz, 2H, —N—CH_{2eq}—C); δ=3.79 ppm, 6H, —COOCH₃); δ=4.60 ppm (s, 2H, Py-CH); δ=7.17 ppm (m, 2H, —CH_{4-pyridine}); δ=7.71 ppm (m, 2H, —CH_{2-pyridine}); δ=7.89 ppm (m, 2H, —CH_{3-pyridine}); δ=8.51 ppm (m, 2H, —CH_{5-pyridine}).

### Example 8

### Aziridination (Catalysis Example)

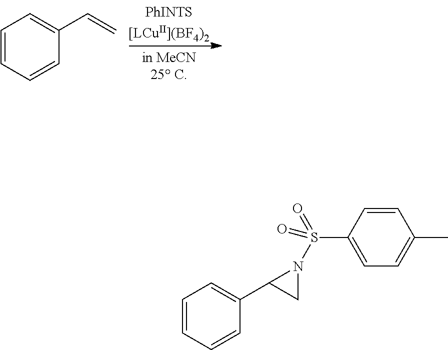

N-Tosyliminophenyliodinane (1 eq, 0.4 mmol, 150 mg), the copper catalyst (5 mol %, 0.02 mmol), and column-treated, degassed styrene (22 eq, 8.7 mmol, 1 ml) were stirred in dry, degassed acetonitrile under nitrogen at 25° C. until the reaction mixture became clear (max. 7 h). The solution was filtered through a short, neutral alumina column, and the column was rinsed with ethyl acetate (20 ml). The solvent was removed under reduced pressure, the residue and the standard anthrone (0.5 eq, 0.2 mmol, 38.8 mg) were dissolved in CDCl₃ (0.6 ml), and the yield of 2-phenyl-1-tosylaziridine was determined by means of ¹H-NMR. The following signals were taken into consideration: anthrone: 4.31 ppm (s, 2H, CH₂), aziridine: 2.97 ppm (d, 1H, ³J_{HH}=7.2 Hz, CH in the three-membered ring trans to Ph).

Yield: 360 mg (0.63 mmol, 36.7%)

An integral ratio of the signals taken into account of 1:1 corresponds to a yield of 100% of aziridination product (0.4 mmol). The TON (turnover number) is calculated from this as follows:

$$TON = \frac{n ( aziridination product )}{n ( catalyst )}$$

With an amount of catalyst of 5 mol % relative to PhINTs, this formula gives a maximum TON of 20.

¹³C-NMR: (CDCl₃, 50.28 MHz)

δ=24.28 ppm (1C, —CH₃); δ=42.92 ppm (1C, N—CH₃); δ=48.60 ppm (2C, —CH₃); δ=52.38 ppm (2C, O—CH₃), δ=52.79 ppm (2C, —C—CH₂—N); δ=60.15 ppm (2C, —CH-Py); δ=61.97 ppm (2C, —CH₂—CH₂); δ=63.17 ppm (2C, N—CH₂—C); =64.98 ppm (2C, —C—COOCH₃); δ=122.84 ppm (2C, —CH_{pyridine/3}); δ=124.19 ppm (2C, —CH_{pyridine/5}) δ=136.14 ppm (2C, —CH_{pyridine/4}) δ=149.36 ppm (2C, —CH_{pyridine/6}) δ=158.47 ppm (2C, —C_{pyridine/2}) δ=169.41 ppm (2C, —COOCH₃); δ=203.46 ppm (1C, CO).

IR spectrum: (KBr disk):

\tilde{v}=3449 cm⁻¹ (b) (H₂O in disk); \tilde{v}=3047 cm⁻¹ (m) (aryl-CH stretching); \tilde{v}=2940 cm⁻¹ (s) (CH₂ and CH₃ stretching); \tilde{v}=2802 cm⁻¹, \tilde{v}=2766 cm⁻¹ (s) (N—CH₃ and N—CH₂ (Bohlmann band) stretching); \tilde{v}=1738 cm⁻¹ (s) (CO vibration); \tilde{v}=1588 cm⁻¹ (w), 1570 cm⁻¹ (m) (aryl-C═C stretching); \tilde{v}=1461 cm⁻¹, \tilde{v}=1432 cm⁻¹ (s) (CH₂ and CH₃ bending); \tilde{v}=1362 cm⁻¹ (m)(CH₃ bending, symmetric)=\tilde{v}758 cm⁻¹ (s)(CH bending, mono-substitution).

Mass spectrum (ESI, MeOH)

m/z: 597.4 (41%) [B4(CH₃OH)(H)]⁺; 565.4 (100%) [B4(H)]⁺.

### Example 9

### Activity of the Copper(II) Bispidone(II) Complex [Cu^{(II)}(B1)(NCCH₃)](BF₄)₂

If [Cu^{(II)}(B1)(NCCH₃)](BF₄)₂ is reacted as an aziridination catalyst by the method of Halfen et al. (cf. J. A. Halfen, J. K. Hallman, J. A. Schultz, J. P. Emerson, Organometallics, 1999, 18, 5435), the results obtained are as listed in table 1.

TABLE 1

| mol % | Styrene:PhINTs | Yield^{a} | t | | V(CH₃CN) |
|---|---|---|---|---|---|
| 5 | 22:1 | 89% | 5 | min | 2 ml |
| 2 | 22:1 | 97% | 8 | min | 2 ml |
| 0.5 | 22:1 | 85% | 40 | min | 2 ml |
| 0.25 | 22:1 | 85% | 3.3 | h | 2 ml |
| 1 | 1:1 | 70% | 28 | min | 0.5 ml |
| 1 | 1:1 | n.d. | >3.5 | h^{b} | 2 ml |

^{a}mol % in respect of PhINTs, T = 25° C.
^{b}Experiment discontinued after 3.5 h owing to excessive duration.

This experiment demonstrates the surprisingly high activity of the metal complex of the invention in the catalytic oxidation of styrene.

### Example 10

### Synthesis of Further Transition Metal Complexes

### 10.1[Co^{II}(B1)]

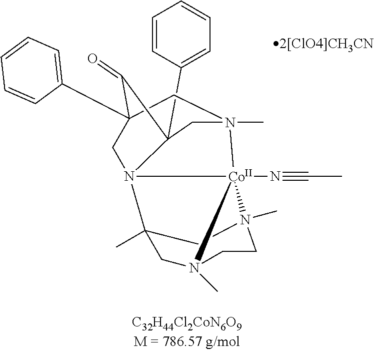

### Batch:

| 1,5-Diphenyl-3,7-(di[1,4,6- | 0.35 | mmol (200 mg) |
|---|---|---|
| trimethyl-1,4-diazacycloheptan-6- | | |
| yl])diazabicyclo[3.3.1]nonan-9-one: | | |
| Co^{II}(ClO₄)₂(H₂O)₆: | 0.38 | mmol (130 mg) |
| Acetonitrile: | 4 | ml |
| Diethyl ether: | 10 | ml |

### Procedure:

In a 50 ml flask, cobalt(II) perchlorate is introduced in 2 ml of acetonitrile. Subsequently B1, likewise in solution in 2 ml of acetonitrile, is added and the mixture is briefly heated. The solution, which has a dark violet coloration, is stirred overnight. Ether diffusion produces pink crystals. Purification takes place by repeated recrystallization from acetonitrile.

Yield: 200 mg (0.25 mmol, 73%).

Appearance: pink crystals.

IR spectrum: (KBr disk):

\tilde{v}=3468 cm⁻¹ (m) (H₂O in disk); \tilde{v}=3065 cm⁻¹ (m) (aryl-CH stretching); \tilde{v}=2977 cm⁻¹ (w), 2933 cm⁻¹ (w) (CH₂ and CH₃ stretching); \tilde{v}=2305 cm⁻¹, \tilde{v}=2277 cm⁻¹, \tilde{v}=2252 cm (w)(CN stretching); \tilde{v}=1744 cm⁻¹ (m) (CO vibration); \tilde{v}=1603 cm⁻¹, \tilde{v}=1505 cm⁻¹ (w) (aryl-C═C vibration); \tilde{v}=1475 cm⁻¹ (m), 1450 cm⁻¹ (m) (CH₂ and CH₃ bending); \tilde{v}=1092 cm⁻¹ (s,b)(ClO₄); \tilde{v}=768 cm⁻¹ (s), 698 cm⁻¹ (s)(CH bending, mono-substitution).

UV-Vis spectroscopy (CH₃CN 3.5 mg in 3 ml)

λ₁=426 nm (ε=27.19/(mol·cm)), λ₂=494 nm (ε=35.17/(mol·cm)), λ₃=525 nm (ε=50.95/(mol·cm)), λ₄=544 nm (ε=55.14 l/(mol·cm)), λ₅=729 nm (ε=19.77/(mol·cm)).

Mass spectrum (ESI, CH₃CN)

m/z: 604.18 (41%) [Co^{II}(B1)(ClO₄)]⁺, 540.21 (100%) [Co^{II}(B1)(Cl)]⁺.

Elemental Analysis (Analysis as CoB1(ClO₄)₂(CH₃CN)₂)

calculated: C, (48.86%); H, (5.64%); N,(10.68%).

found: C, (48.66%); H, (5.55%); N, (10.60%).

### 10.2 [Co^{II}(B3)](BF₄)₂

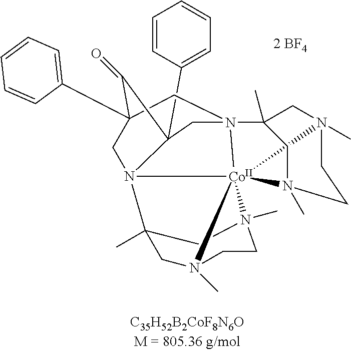

### Batch:

| 1,5-Diphenyl-3,7-(di[1,4,6- | 0.35 | mmol (200 mg) |
|---|---|---|
| trimethyl-1,4-diazacycloheptan-6- | | |
| yl])diazabicyclo[3.3.1]nonan-9-one: | | |
| Co^{II}(BF₄)₂(H₂O)₆: | 0.38 | mmol (130 mg) |
| Acetonitrile: | 4 | ml |
| Diethyl ether: | 10 | ml |

### Procedure:

In a 50 ml flask, cobalt(II) tetrafluoroborate is introduced in 2 ml of acetonitrile. Subsequently B3, likewise in solution in 2 ml of acetonitrile, is added and the mixture is briefly heated. The solution, which has a pink coloration, is stirred overnight. Ether diffusion produces pink crystals. Purification takes place by recrystallization from acetonitrile.

Yield: 200 mg (0.25 mmol, 71%).

Appearance: pink crystals.

IR spectrum: (KBr disk):

\tilde{v}=3435 cm⁻¹ (m) (H₂O in disk); \tilde{v}=3028 cm⁻¹ (w) (aryl-CH stretching); \tilde{v}=2976 cm⁻¹ (w) (CH₂ and CH₃ stretching); \tilde{v}=1742 cm⁻¹ (m) (CO vibration); \tilde{v}=1630 cm⁻¹ (w) (aryl-C═C stretching); \tilde{v}=1481 cm⁻¹ (m), 1447 cm⁻¹ (m) (CH₂ and CH₃ bending); \tilde{v}=1057 cm⁻¹ (s,b) (BF vibration); \tilde{v}=772 cm⁻¹ (s), 703 cm⁻¹ (s) (CH bending, mono-substitution).

UV-Vis spectroscopy (CH₃CN 12.4 mg in 3 ml)

λ₁=471 nm (ε=9.07/(mol·cm)), A₂=567 nm (ε=25.66/(mol·cm)), λ₃=864 nm (ε=4.26/(mol·cm)).

Mass spectrum (ESI, CH₃CN)

m/z: 315.6 (32%) [Co^{II}(B3)]²⁺, 650.3 (100%) [Co^{II}(B3)(H₂O)(H)]⁺.

Cyclic voltammogram (vs. Ag/AgNO₃, T=25° C. in CH₃CN abs.)

E_{1/2}=+1504 mV.

Elemental Analysis

calculated: C, (52.20%); H, (6.51%); N, (10.43%).

found: C, (52.18%); H, (6.74%); N, (10.48%).

### 10.3 [Z^{II}(B3)]ZnCl₄

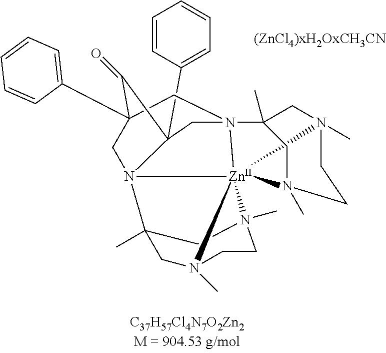

### Batch:

| 1,5-Diphenyl-3,7-(di[1,4,6- | 0.52 | mmol (300 mg) |
|---|---|---|
| trimethyl-1,4-diazacycloheptan-6- | | |
| yl])diazabicyclo[3.3.1]nonan-9-one: | | |
| Zn^{II}Cl₂: | 0.62 | mmol (85 mg) |
| Acetonitrile: | 24 | ml |

### Procedure:

In a 100 ml flask, zinc(II) chloride in solution in 12 ml of acetonitrile is introduced. Subsequently B3, likewise in solution in 12 ml of acetonitrile, is added and the mixture is briefly heated. The solution, which is colorless but slightly turbid, is stirred overnight. The white precipitate is isolated and washed repeatedly with approximately 5 ml of acetonitrile. The white solid is dried under reduced pressure.

Yield: 209 mg (0.23 mmol, 45%).

Appearance: white solid.

IR spectrum: (KBr disk):

\tilde{v}=3461 cm⁻¹ (m) (H₂O in disk); \tilde{v}=3058 cm⁻¹ (w) (aryl-CH stretching); \tilde{v}=2971 cm⁻¹ (w), \tilde{v}=2950 cm⁻¹ (w), \tilde{v}=2926 cm⁻¹ (w) (CH₂ and CH₃ stretching); \tilde{v}=1740 cm⁻¹ (m)(CO vibration); \tilde{v}=1637 cm⁻¹ (w)(aryl-C═C stretching); \tilde{v}=1479 cm⁻¹ (m), 1446 cm⁻¹ (m) (CH₂ and CH₃ bending); \tilde{v}=766 cm⁻¹ (s), 696 cm⁻¹ (s) (CH bending, mono-substitution).

Mass spectrum (ESI, CH₃CN)

m/z: 318.2 (1%) [Zn^{II} (B3)]²⁺, 573.4 (100%) [(B3)(H)]⁺, 671.3 (26%) [Zn^{I} (B3) Cl]⁺.

Elemental Analysis

calculated: C, (49.13%); H, (6.35%); N, (10.84%).

found: C, (49.26%); H, (6.24%); N, (11.10%).

### 10.4 [Fe^{II}(B3)]FeCl₄

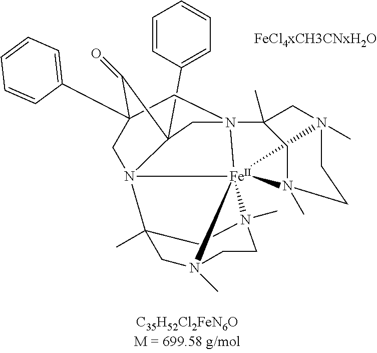

### Batch:

| 1,5-Diphenyl-3,7-(di[1,4,6- | 0.43 | mmol (244 mg) |
|---|---|---|
| trimethyl-1,4-diazacycloheptan-6- | | |
| yl])diazabicyclo[3.3.1]nonan-9-one: | | |
| Fe^{II}Cl₂: | 0.32 | mmol (41 mg) |
| Acetonitrile (abs.): | 40 | ml |

### Procedure:

In a glovebox/under argon inert gas, in a 100 ml flask, iron(II) chloride dissolved with 20 ml of acetonitrile (abs.) is introduced. Subsequently B3, likewise in solution in 20 ml of acetonitrile (abs.), is added; the mixture is stirred overnight. This produces a yellowish precipitate. This precipitate is isolated and washed repeatedly with about 10 ml of acetonitrile (abs.). The yellowish solid is dried under reduced pressure and for further measurements is handled under inert gas.

Yield: 97 mg (0.14 mmol, 23%).

Appearance: yellow solid.

IR spectrum: (KBr disk):

\tilde{v}i=3455 cm⁻¹ (m) (H₂O in disk); \tilde{v}=3059 cm⁻¹ (w) (aryl-CH stretching); \tilde{v}=2960 cm⁻¹ (w), \tilde{v}=2926 cm⁻¹ (w), \tilde{v}=2884 cm⁻¹ (w) (CH₂ and CH₃ stretching); \tilde{v}=1738 cm⁻¹ (m)(CO vibration); \tilde{v}=1635 cm⁻¹ (w)(aryl-C═C stretching); \tilde{v}=1478 cm⁻¹ (m), 1446 cm⁻¹ (m) (CH₂ and CH₃ bending); \tilde{v}=765 cm⁻¹ (s), 697 cm⁻¹ (s) (CH bending, mono-substitution).

Mass spectrum (ESI, CH₃OH)

m/z: 573.4 (100%) [(B3)(H)]⁺, 663.3 (1%) [Fe^{II}(B3)(Cl)]⁺, 699.3 (1%) [Fe^{II} (B3)(Cl₂)(H)]⁺.

Elemental Analysis

calculated: C, (50.19%); H, (6.49%); N, (11.07%).

found: C, (50.40%); H, (6.45%); N, (11.02%).

1. A bispidone ligand of formula (1): 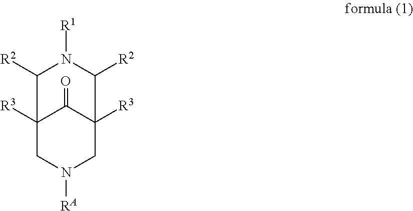
in which
the radical R^{A} is selected from a group of one of the formulae (2a) to (2d): 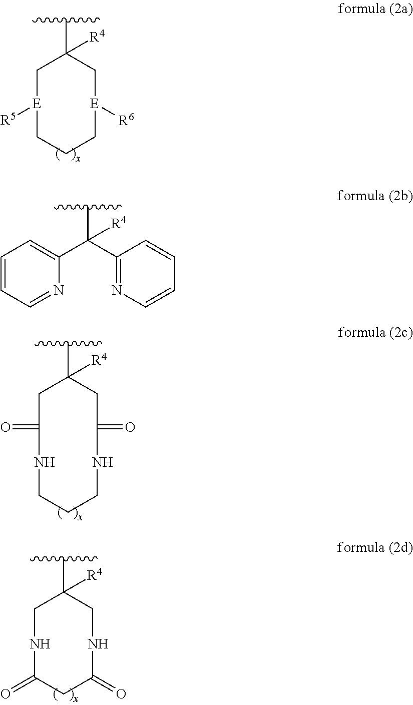
in which E is selected from N or P, and x is an integer from 0 to 5, the radical R¹ is selected from hydrogen, straight-chain or branched-chain (C₁₋₁₂) alkyl radicals, (C₃₋₈) cycloalkyl radicals, (C₅₋₁₂) aryl or heteroaryl radicals, (C₆₋₁₂) alkaryl or alkheteroaryl radicals, or a group of the formula (2a) to (2d) defined as above, with E and x being defined as above,
both radicals R² are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals,
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
the radical R⁴ is selected from hydrogen, straight-chain or branched-chain (C₁₋₁₂) alkyl radicals, (C₃₋₈) cycloalkyl radicals, (C₆₋₁₂) aryl or (C₅-C₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

2. The bispidone ligand as claimed in claim 1, in which the radical R^{A} is a group of the formula (2a): 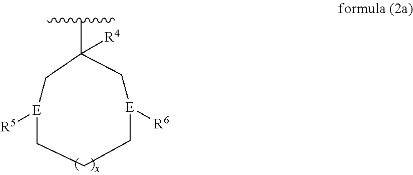
the radical R¹ is a straight-chain or branched-chain (C₁₋₆) alkyl radical, a (C₆₋₁₂) alkheteroaryl radical or a group of the above-defined formula (2a),
both radicals R² are selected each independently of one another from hydrogen or a (C₆₋₁₂) aryl or (C₅₋₁₂) heteroaryl radical,
both radicals R³ are selected each independently of one another from (C₁₋₆) aryl or heteroaryl groups or carboxylic acid groups or derivatives derived therefrom, selected from esters, amides, and peptides,
the radical R⁴ is selected from hydrogen, straight-chain or branched-chain (C₁₋₁₂) alkyl radicals or (C₃₋₈) cycloalkyl radicals, and
both radicals R⁵ and R⁶ are selected each independently of one another from hydrogen, straight-chain or branched-chain (C₁₋₁₂) alkyl radicals or (C₃₋₈) cycloalkyl radicals, and
where E is selected from N or P, and x is an integer from 0 to 5.

3. The bispidone ligand as claimed in claim 1, in which
the radical R^{A} is a group of the formula (2a): 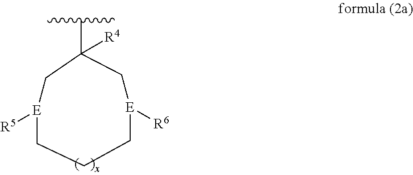
the radical R¹ is methyl, picolinyl or a group of the formula (3): 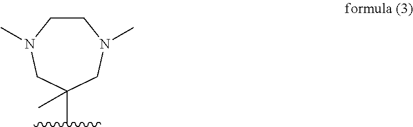
both radicals R² are hydrogen or pyridinyl groups,
both radicals R³ are phenyl or methanoic acid methyl ester groups, and the radicals R⁴ to R⁶ are methyl, and
where E is N and x=0.

4. A process for preparing the bispidone ligand defined in claim 1, where the group R² represents hydrogen, comprising the steps of:
(a) the reacting of a compound of one of the formulae (4a) to (4d): 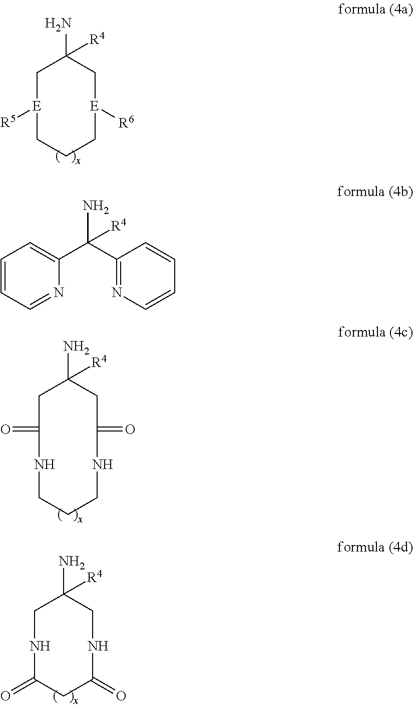
in which
E is selected from N or P, x is an integer from 0 to 5,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals,
with formaldehyde and with an acetone derivative of the formula (5): 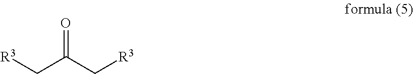
in which
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
to form a piperidone intermediate of the formula (6): 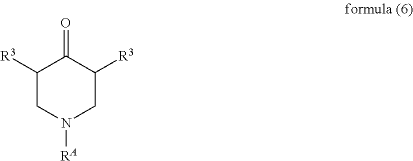
in which
the radicals R^{A} and R³ are defined as above, and
(b) the reacting of the piperidone intermediate of the formula (6) with formaldehyde and with an amine of the general formula H₂N-R¹, in which
the radical R¹ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, (C₆₋₁₂) alkylaryl or alkheteroaryl groups, or a group of the formula (2a) to (2d) defined as above, where E and x are defined as above, and
where the radicals R³ to R⁶ are defined as above.

5. The process for preparing the bispidone ligand defined in claim 1, in which the radical R¹ is a group of the formula (2a) 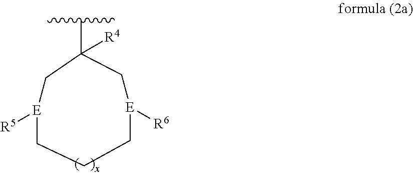
and both radicals R² are hydrogen, comprising the reacting of a compound of the formula (4a): 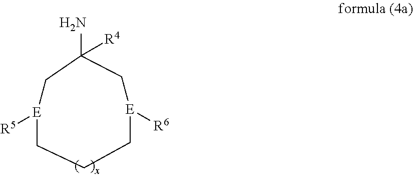
with formaldehyde and with an acetone derivative of the formula (5): 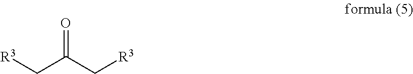
in which
both radicals R³ are selected each independently of one another from hydrogen, straight-chain or branched-chain (C₁₋₁₂) alkyl radicals, (C₃₋₈) cycloalkyl radicals, (C₈₋₁₂) aryl or (C₅₋₁₂) heteroaryl radicals or carboxylic acid groups or derivatives derived therefrom, selected from esters, amides, and peptides,
the radicals R⁴ to R⁶ are selected each independently of one another from hydrogen or straight-chain or branched-chain (C₁₋₈) alkyl radicals, and where E is selected from N or P, and x is an integer from 0 to 5.

6. The process for preparing the bispidone ligand as defined in claim 1, comprising the reacting of a compound according to one of the above-defined
formulae (4a) to (4d), in which
E is selected from N or P and x is an integer from 0 to 5,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals,
with formaldehyde and with a compound of the formula (7): 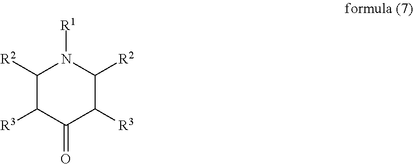
in which
the radical R¹ is selected from hydrogen, straight-chain or branched-chain (C₁₋₁₂) alkyl radicals, (C₃₋₈) cycloalkyl radicals, (C₅₋₁₂) aryl or heteroaryl radicals, (C₆₋₁₂) alkaryl or alkheteroaryl groups, or a group of the formula (2a) to (2d) as defined above, where E and x are defined as above,
both radicals R² are selected each independently of one another from hydrogen, straight-chain or branched-chain (C₁₋₁₂) alkyl radicals, (C₃₋₈) cycloalkyl radicals, and (C₆₋₁₂) aryl or (C₅₋₁₂) heteroaryl radicals,
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

7. A process of using the bispidone ligand as claimed in claim 1 for selective separation of metal ions, for preparation of metal complexes for catalytic oxidation of unsaturated compounds, for catalytic bleaching or for diagnosis and/or therapy of tumor diseases.

8. A metal complex comprising a bispidone ligand as claimed in claim 1, the metal being selected from Mn, Cu, Fe, Co, Ti, V, Mo, W, Tc, In, Ga, Y, Re or the rare earth metals.

9. The metal complex as claimed in claim 8, the metal being a radioactive nuclide.

10. A process for preparing a metal complex comprising the bispidone ligand as claimed in claim 1 being reacted with a metal salt solution of the corresponding metal at a temperature in the range from 20 to 100° C., the metal being selected from Mn, Cu, Fe, Co, Ti, V, Mo, W, Tc, In, Ga, Y, Re or the rare earth metals.

11. A process of using the metal complex as claimed in claim 8 in catalytic oxidation of unsaturated compounds, in catalytic bleaching, or in the diagnosis and/or therapy of tumor diseases.

12. A compound having the formula (6): 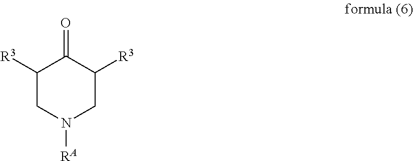
in which
the radical R^{A} is selected from a group of one of the formulae (2a) to (2d): 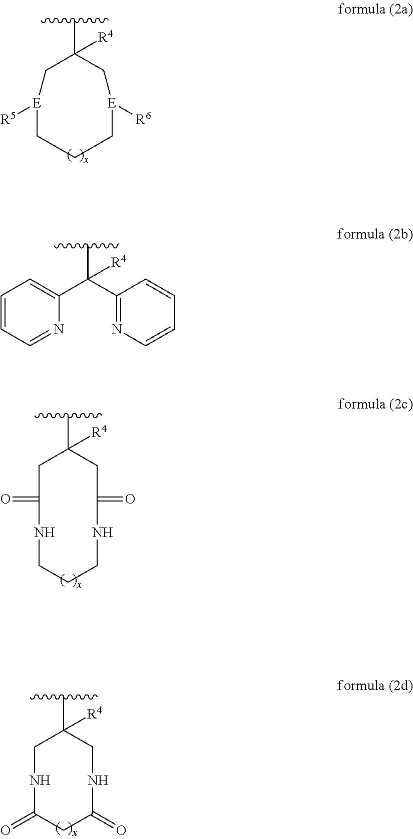
in which E is selected from N or P, and x is an integer from 0 to 5,
both radicals R³ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals or carboxylic acid groups or derivatives derived therefrom, selected from esters, amides, and peptides,
the radical R⁴ is selected from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals, and optionally
both radicals R⁵ and R⁶ are selected each independently of one another from hydrogen, straight-chain or branched-chain (C₁₋₁₂) alkyl radicals, (C₃₋₈) cycloalkyl radicals, (C₆₋₁₂) aryl or (C₅₋₁₂) heteroaryl radicals.

13. The process as claimed in claim 10, the metal being a radioactive nuclide.

14. The process as claimed in claim 9, the metal being a radioactive nuclide.
